(19) United States
(12) Patent Application Publication
(10) Pub. No.: US 2023/0347004 A1
(43) Pub. Date: Nov. 2, 2023

(54) Liner for Home Decor Wax Warmers

(71) Applicant: Rebecca Ann Treadway Fancher, West Monroe, LA (US)

(72) Inventor: Rebecca Ann Treadway Fancher, West Monroe, LA (US)

(21) Appl. No.: 18/219,019

(22) Filed: Jul. 6, 2023

Related U.S. Application Data
(63) Continuation-in-part of application No. 17/301,307, filed on Mar. 31, 2021, now Pat. No. 11,745,918.
Application No. 17/301,307 is a continuation-in-part of application No. 16/231,093, filed on Dec. 21, 2018, now abandoned.

(51) Int. Cl. A61L 9/03

(52) U.S. Cl. CPC A61L 9/03; A61L 2209/133

(57) ABSTRACT

Scented wax often melted in a decorative heated warmer for aromatherapy, while pleasant and therapeutical, creates a mess while changing scents. Scented wax that is melted, then cooled in the warming tray of a wax warmer is difficult to remove. The present invention is liner that fits into the inner part of a wax warming dish and is made to hold scented wax and prevent the melted wax from coming in contact with the warming dish. This liner acts as a barrier between the scented wax and the warming tray of the wax warmer and makes it easy to remove the scented wax from the warming dish, without making a mess while keeping the wax warmer clean.

This invention is a CIP of application Ser. No. 17/301,307.

### BACKGROUND OF THE INVENTION

Creating an ambient atmosphere with scents has been recorded through the ages, even in ancient times, with herbs and spices being used to mask odors or a particular wood being burned to create a reverent feeling of calm and worship.

Throughout the centuries, various means of transmitting these aromas have been implemented, such as the use of incense, and more recently, wax. Waxes are commonly produced from petroleum, tallow, bees wax or soy products and can vary in density and hardness. Scents are added to present a pleasant environment for the enjoyment of the user. Often these waxes are burned, but sometimes, they are melted at a lower temperature to allow the scent to linger longer and without the danger of an open flame.

In recent decades, wax warmers for decorative purposes have become popular, and small wax pieces, typically called ‘tarts’ have been created to use within the warming tray of the wax warming unit. Wax warmers can come in a variety of sizes and shapes and are usually made into appealing designs to match interior décor. These decorative wax warmers are used for enjoyment purposes only, rather than the vats of wax used for medicinal and therapeutic purposes of wax therapy for the hands and feet of an individual, therefore are made to hold only a small amount of wax, often not even an ounce.

Decorative wax warmers consist of a lower chamber that hosts a heating mechanism, such as a small votive candle, a lower wattage light bulb or a heating element. The temperature from the candle, bulb or element heats the bottom of a warming tray, situated above. Scented wax is then placed in the warming tray and melts within, allowing the aroma to spread into the atmosphere surrounding the wax warmer.

The temperature of the warming tray of decorative wax warmers is generally low, as the purpose is to gently melt the wax rather than to cook the wax, and ranges from approximately 130 degrees to 250 degrees. Lower temperatures will not melt the wax adequately, and higher temperatures will use up the wax properties quicker and be dangerous to the touch.

The biggest drawback to these decorative wax warmers is cleaning the warming tray to change the wax to a different scent. Once wax has been melted from its original form of a square, chip or block, it fills the warming tray and takes the shape of the tray. A variety of ways have been devised to remove the wax which are described below:

1) Cold changing: The warming tray filled with wax which has cooled and hardened is placed in freezing temperatures, such as in the freezer portion of a refrigerator, and allowed to sit until frozen, approximately 30 minutes. The tray is then removed from the freezer and the wax can be pushed out of the warming tray in most cases.

2) Sharp edge: Plastic pieces, such as a corner of plastic packaging or a plastic cooking spatula are used to loosen the edges of the wax cooled inside the warming tray, working around the edges of the wax, then lifting out. This usually leaves pieces of wax in the warming tray to be cleaned out afterwards.

3) Heat and Scoop: Allow the warming tray to cool so the wax becomes solid. Then warm for a few minutes to loosen the wax and scoop it out with fingers to remove the softened wax. If wax is not removed soon enough, it becomes liquid again and the process has to be repeated until the wax is removed at the appropriate time.

4) Warm Wax Removal: Allow the wax to cool approximately 20-30 minutes, then push the softened cooled wax out with fingers and wipe out the residue. The residue will flake and be quite messy and very hard to remove.

5) Hot Dog Method: Pour melted wax straight into a trash or garbage container. Be sure to position the container close to the wax warmer, as movement may spill the liquid wax and be quite messy to clean up or may burn the fingers when holding the warming tray. Wipe the residue to finish cleaning the warming tray.

6) Cotton Ball: Soak up the liquid wax from the warming tray by placing absorbent material directly into the wax while it is still hot. Removing the wax-soaked material may be hot to the touch or drip while being transported to a trash container.

Given the difficulties and dilemma of removing scented wax from the warming tray of a wax warmer, most wax warmers go unused and non-effective. The refusal to clean a messy wax warmer is common and unused scented wax purchased for the purpose of use in the wax warmer is wasted indefinitely.

The present invention allows those who wish to use their decorative wax warmers efficiently to safely maintain the cleanliness of their warming trays by placing the scented wax within a liner which is placed in the warming tray of the wax warmer. The liner acts as a barrier between the wax and the warmer and melts the wax within effectively and without inhibiting the “throw” or scent emissions of the wax. When cooled, the entire liner containing the melted wax can be easily removed by picking it up with fingers, leaving the wax warming tray as clean and good as new, ready to use another piece of scented wax.

The present invention goes a step further in encouraging re-use of the liner by easily removing the hardened wax from the liner itself.

As a continuation of the previous patent application Ser. No. 16/231,093 and approved application Ser. No. 17/301,307, this invention provides details of the material content of the liner as well as presents a solution to the removal of the hardened wax from the liner itself.

While the present invention may be manufactured of various materials, including metal, paper, metallic coated paper, silicone, polyethylene or other polymers, the preferred material is a polymer with transparent, or clear, properties as to not distract from the decorative appearance of the wax warmer itself. The thickness of the material should be adequate to withstand the heat of the warming tray, yet not affect the “throw” or scent emissions of the scented wax. Given the range of temperatures of the heating trays of typical decorative wax warmers, this thickness ideally would be between 10 ml to 15 ml, and perhaps up to 20 ml allowing thorough heating for the scented wax to melt within and not melt the liner itself.

Preserving the integrity of the liner is important for the sake of re-using the liner for subsequent uses. Removing the scented wax from the liner after use is vital to this goal. While the liner itself preserves the cleanliness of the warming tray of the wax warmer, the ability to remove the cooled wax from the liner is a different matter.

The present invention allows for the contours and bottom ridge of the previous application Ser. No. 17/301,307 while presenting a solution to the removal of the scented wax by including a centrally located button or bubble extending downward and outward from the recessed portion of the liner base to aid in pushing out the hardened wax, allowing the liner to easily be re-used.

### FIELD OF INVENTION

The popularity of scented wax in home décor has grown over the last several decades. Scented wax nallows an ambient slow release of aroma without burning, which can change molecular properties of wax as well as the scent mixed into the wax. When the wax is warmed it allows the scent to permeate the atmosphere surrounding the wax warmer without the difficulties of having to continuously watch it in progress, as one would with having to mind a candle or open flame.

Wax warmers have become a popular part of home décor and are made in a variety of shapes to provide pleasing and appealing additions to everyday decorating as well as specialty designs for holidays and special occasions. Wax warmers consist of a base with some kind of heating mechanism, which could be an actual candle, called a votive candle, which are very small and would fit within the base of a warmer, a light bulb with low wattage chosen specifically for melting wax or an electrical element to directly provide heat to the warmer. The heat provided to the base of the warmer reaches the warming tray of the wax warmer, which is located above the heating method which melts the wax within the tray.

Until now, cleaning the wax from a warming tray of a wax warmer has been difficult, messy and even dangerous. Various solutions have been contrived to remove hardened wax, and often the wax warmer is abandoned for use after futile attempts at cleaning have failed.

The present invention allows the user to enjoy using a wax warmer for decorative and aromatic purposes by providing a simple way of removing the molten or hardened wax while keeping the wax warming tray clean and free of ugly wax residue.

### SUMMARY OF THE INVENTION

The present invention is a liner made to fit inside the warming tray of a decorative wax warmer, creating a barrier between the scented wax held within and the surface of the warming tray. The liner prevents the scented wax from touching the surface of the warming tray, which allows the warming tray to remain clean and aesthetically pleasing as well as ready for the next scented wax use.

The present invention is preferred to be made of a clear polymer, between 10 ml and 20 ml in thickness as to absorb heat from the heating elements of the wax warmer to transfer through the surface of the warming tray and melt the scented wax within the liner without melting the liner itself. The liner should be able to sustain temperatures up to 250 degrees. The clarity of the polymer is clear and transparent, as to be invisible and not distracting from the decorative appearance of the wax warmer. The degree of clarity is defined by the type of polymer, but transparent, without color is preferred. Preferred polymers are polyethylene and polypropylene, which require either thermoforming or injection molding. These polymers will allow the “throw” or scent from the wax to maintain its original properties without inhibiting them, as would silicone or other materials such as paper.

The diameter of the liner body ranges from 1.5″ to approximately 4″ which is the general diameter range for the warming trays of decorative wax warmers from smaller wall plug-in units to larger countertop versions. Other geometric shapes can be adapted as well.

The liner comprises a bottom portion which has a ridged feature as described in the CIP application Ser. No. 17/301,307, protruding downward encircling the bottom edge of the liner. A recessed portion completes the bottom of the liner. A wall rising up from the bottom of the liner is formed together in one piece with the bottom and contains levels or tiers that succeedingly extend upward and outward so that the bottom circumference of the liner is smaller than the top edge circumference of the surrounding wall and each level or tier is slightly larger in circumference than the one below it. An encircling flat ledge or flange extending outward from the top edge of the surrounding wall provides a way to remove the liner from the warming tray and helps stabilize the liner within the warming tray.

A round button or bubble shaped extension is centrally located and protrudes downward from the recessed liner bottom and extends to the depth of the ridge, so the tip of the button or bubble extends no further than the surrounding ridge itself to allow the liner to sit flatly on the surface of the warming tray. The ridge sits on the surface of the wax warming tray, providing an air cushion between the liner bottom and the warming tray surface, which allows for even heating and prevents condensation from forming which can cause the liner to stick to the warming tray. The button or bubble can be circular in shape and may be arched and rounded or have a flat tip in a protrusion equal to or less than the protrusion of the encircling ridge. The button or bubble is smaller than the recessed portion from which it extends and is centrally located.

When scented wax is heated, then cooled inside the liner and the user wishes to remove the cooled wax for further use of the liner, the button or bubble centrally located extending outward and downward from the bottom of the liner allows the user to push the cooled wax out by pushing the bubble or button upward and inward toward the interior of the liner. Once the wax has loosened, it can be easily removed and safely discarded and the liner can be re-used for another piece of scented wax. This method allows the liner to be used indefinitely and encourages use of the wax warmer and scented wax in general by making the wax removal process simple, efficient and effective.

### DESCRIPTION OF RELATED ART

There have been several patent applications with similar concepts to this invention that have been identified since this author's first application Ser. No. 16/231,093 was published. US 2019/0359386 A1, Siva Jaklinki's application for a “Disposable Liner Container for Wax Warmer” presents a similar concept preferencing metal as a material and “wings” for grasping and removing the piece from the warming tray of a wax warmer. His invention consists of a flat bottom portion with a flat surrounding wall with sides that extend upward with extended “wings” as portions that continue upward from opposing sides of the wall. The drawbacks of his proposed invention are detailed in the CIP of this author's original application, application Ser. No. 17/301,307.

A further view was discovered after the CIP was filed for this author's original application of Anderson et al (US 2020/0205235) who claims a similar concept of a liner for a wax warmer formed of silicone and releases the wax within by reversing the flexible, rubbery material inside outwards and dumping the wax out. The drawbacks of this concept are that silicone retains the properties of the melted wax which will prohibit the “throw” of the scent of the warming wax. Removing the wax by flipping the object inside outwards may result in splashing hot wax if wax is not cooled efficiently, which could be a danger to the user. The act of doing so also may render the device ineffective by stretching or tearing of the material.

This author's previous applications mentioned above have provided detailed descriptions for the concept of a liner made for the warming tray of a decorative wax warmer to act as a barrier between the scented wax placed within and the interior of the warming tray itself. The liner of the CIP application Ser. No. 17/301,307 specifies a bottom portion with surrounding ridge extending downward with attached side wall leading upward in “steps” to provide contours for stability purposes and to aid in the removal of hardened wax within the liner itself. An outward formed ledge encircles the top edge of the surrounding wall which provides both a means of removal from the warming tray as well as helping to maintain the upright position of the liner as it is placed in the warming tray.

### DETAILED DESCRIPTION OF THE DRAWINGS

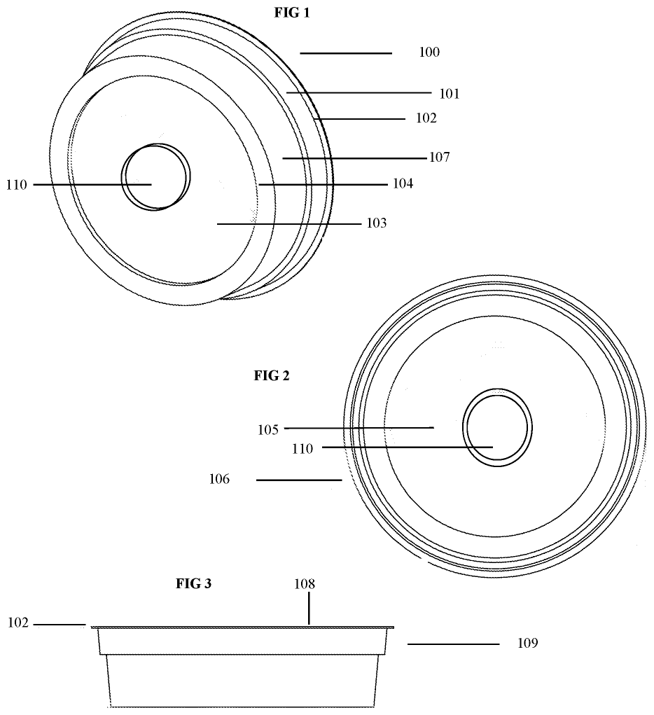

FIG. 1 shows a bottom side view of the invention as it would look three dimensionally 100. An upper tier wall or level 101 is formed into the side of the liner structure at the open top edge where it is met with an outwardly formed ledge or flange 102 that surrounds the top perimeter of the wall and is fashioned perpendicularly to the side of the wall. The recessed portion of the bottom of the liner structure 103 is surrounded by the raised ridge 104 that encircles the outer edge of the bottom circumference. The bottom side wall 107 rises from the base of the ridge edge and extends upward at a slight angle, making the bottom circumference slightly smaller than the top of the wall. Each succeeding tier or level is slightly larger than the one below it. The button or bubble 110, located centrally at the bottom of the liner structure and formed outward from the recessed portion 103 of the bottom is smaller than the recessed portion and does not protrude past the ridge 104 that surrounds the circumference of the bottom of the liner.

FIG. 2 shows an inner view 105 of the recessed portion 103 of the bottom of the liner as looking downward from the top opening 108 of the liner structure. This angle is the top view 106 of the structure from the open edge 108, looking down inward.

FIG. 3 is the side view 109 of the liner structure indicating the top opening 108 and the perpendicularly formed flange or ledge 102.

What I claim as my invention is the following:

1. A liner for Home Décor Wax Warmers comprising:
A single piece of polymer, said piece consists of a bottom piece, a tiered side wall and an top opening;
Said piece comprising a bottom consisting of an outer perimeter; a continuous symmetric ridge and a center portion;
Whereas said continuous, symmetric ridge extends downward entirely around said outer perimeter of said bottom, and wherein said continuous, symmetric ridge extends inward from said outer edge toward said center portion of said bottom;
And wherein said center portion of said bottom is elevated above said continuous, symmetric, downward ridge and contains an outwardly protruding centrally positioned button or bubble smaller than said elevated center portion and not protruding past the limits of said ridge;
Said tiered side wall consisting of a single tier, a bottom section located below said single tier, and an upper section located above said single tier, said upper section and said lower section being angled outward from said outer perimeter of said bottom and said single tier consisting of a section extending parallel to said center portion of said bottom, and said top which is parallel to said center portion of said bottom, and said top which is parallel to said center portion of said bottom and which has an outer perimeter larger than said outer perimeter of said bottom, and wherein said top consists of an edge and a flange formed continuously and outwardly from said edge and wherein said flange is parallel to said center portion of said bottom.

2. Said polymer of claim 1 is of a clear, colorless and translucent grade between 10 ml and 20 ml in thickness and able to withstand temperatures up to 250 degrees for extended amounts of time.

3. Said liner has a circumference up to 4 inches at the top opening.
